(19) United States
(12) Patent Application Publication
(10) Pub. No.: US 2021/0341381 A1
(43) Pub. Date: Nov. 4, 2021

(54) Corrosivity Evaluation Device and Method Thereof

(71) Applicant: Nippon Telegraph and Telephone Corporation, Tokyo (JP)

(72) Inventors: Shingo Mineta, Musashino-shi, Tokyo (JP); Shota Oki, Musashino-shi, Tokyo (JP); Mamoru Mizunuma, Musashino-shi, Tokyo (JP); Masayuki Tsuda, Musashino-shi, Tokyo (JP); Takashi Sawada, Musashino-shi, Tokyo (JP)

(21) Appl. No.: 17/280,278

(22) PCT Filed: Sep. 13, 2019
(86) PCT No.: PCT/JP2019/036202

§ 371 Date: Mar. 26, 2021

(30) Foreign Application Priority Data
Sep. 27, 2018 (JP) 2018-181553

(51) Int. Cl. G01N 17/00; G01N 17/04; G01N 33/2045

(52) U.S. Cl. CPC G01N 17/006; G01N 33/2045; G01N 17/04

(57) ABSTRACT

To provide a corrosivity evaluation device that quantitatively evaluates corrosivity that represents an extent to which metal is corroded by an environment. A corrosivity evaluation device evaluates corrosivity that represents an extent to which metal is corroded by an environment, the device including: an electrode unit containing at least one type of the metal by being placed in the environment; a measurement unit adapted to measure a corrosion rate of the metal or a value related to the corrosion rate of the metal during one cycle of change in water content of the environment, the measurements being taken from the one cycle of change; and a calculation unit adapted to calculate a corrosion amount of the metal or a value related to the corrosion amount of the metal from the value measured by the measurement unit.

### TECHNICAL FIELD

The present invention relates to a corrosivity evaluation device and corrosivity evaluation method that evaluate corrosivity that represents the extent to which metal is corroded by an environment.

### BACKGROUND ART

Infrastructure equipment that supports our life comes in various types and is provided in vast quantities. In addition, the infrastructure equipment is exposed not only to urban environments but also to various environments of mountainous areas, coastal areas, hot-spring areas, cold regions, and the like. For maintenance of infrastructure equipment exposed to such various environments, it becomes necessary to keep track of the current state of deterioration by means of inspection and operate the infrastructure equipment efficiently based on a predictive estimation technique.

For example, there is a lot of infrastructure equipment exposed to atmospheric environments on the ground. By being weather-beaten, such infrastructure equipment is corroded at rates corresponding to respective environments.

Also, infrastructure equipment installed underwater is corroded at a rate peculiar to the environment. Also, there is a lot of underground equipment used by being partially or entirely buried underground as typified by steel pipe columns, support anchors, underground steel pipes, and the like.

As standards for evaluating the corrosivity of soil in which underground equipment is buried, for example, ANSI (American National Standards Institute) and DVGW (Deutscher Verein des Gas-und Wasserfaches: German Technical and Scientific Association for Gas and Water) standards are known (Non-Patent Literature 1).

### CITATION LIST

### Non-Patent Literature

Non-Patent Literature 1: Satomi Tsunoda, et al. “Some Problems for Evaluating Soil Aggressivity,” Corrosion Engineering, Vol. 36, pp. 168-177 (1987)

### SUMMARY OF THE INVENTION

### Technical Problem

Both ANSI and DVGW standards prescribe methods for measuring environmental factors, such as resistivity, pH, and water content, contributing to corrosion, with regard to the soil whose corrosivity is desired to be evaluated and comprehensively evaluating results of the measurements taken together. However, the evaluations are no more than qualitative, and it is difficult to perform quantitative evaluations needed to be used, for example, for deterioration prediction. It is also pointed out that the results thus obtained often do not square with reality

### (Non-Patent Literature 1).

That is, the current state of affairs has a problem in that there is no device or method capable of quantitatively evaluating corrosivity of an environment in which metal is placed.

The present invention has been made in view of the above problem and has an object to provide a corrosivity evaluation device and corrosivity evaluation method capable of quantitatively evaluating corrosivity of an environment in which metal is placed.

### Means for Solving the Problem

According to one aspect of the present invention, there is provided a corrosivity evaluation device that evaluates corrosivity that represents an extent to which metal is corroded by an environment, the device comprising: an electrode unit containing at least one type of the metal by being placed in the environment; a measurement unit adapted to measure a corrosion rate of the metal or a value related to the corrosion rate of the metal during one cycle of change in water content of the environment, the measurements being taken from the one cycle of change; and a calculation unit adapted to calculate a corrosion amount of the metal or a value related to the corrosion amount of the metal from the value measured by the measurement unit.

According to one aspect of the present invention, there is provided a corrosivity evaluation method performed by the corrosivity evaluation device, the method comprising: a measurement step of measuring a corrosion rate of the metal or a value related to the corrosion rate of the metal during one cycle of change in water content of an environment in which at least one type of the metal is placed, the measurements being taken from the one cycle of change; and a calculation step of calculating a corrosion amount of the metal or a value related to the corrosion amount of the metal from the value measured in the measurement step.

### Effects of the Invention

The present invention can quantitatively evaluate corrosivity of an environment in which metal is placed.

### BRIEF DESCRIPTION OF DRAWINGS

FIG. 1 is a block diagram showing an exemplary functional configuration of a corrosivity evaluation device according to an embodiment of the present invention.

FIG. 2 is a diagram showing an operation flow of the corrosion rate estimation device shown in FIG. 1.

FIG. 3 is a diagram schematically showing a relationship between rainfall and soil moisture percentage.

FIG. 4 is a diagram schematically showing a relationship between rainfall and a corrosion rate of metal in soil.

FIG. 5 is a diagram schematically showing a Nyquist diagram.

FIGS. 6(a) and 6(b) are diagrams showing an example of equivalent circuits assumed in calculating charge transfer resistance.

FIGS. 7(a) and 7(b) are diagrams showing an example of equivalent circuits assumed in calculating charge transfer resistance.

FIG. 8 is a diagram schematically showing a relationship between time and a value (1/Rct) proportional to a corrosion rate.

FIG. 9 is a diagram schematically showing an example of a container unit.

FIG. 10 is a diagram schematically showing another example of the container unit.

### DESCRIPTION OF EMBODIMENTS

Embodiments of the present invention will be described below with reference to the drawings. In plural drawings, the same components are denoted by the same reference signs and redundant description thereof will be omitted.

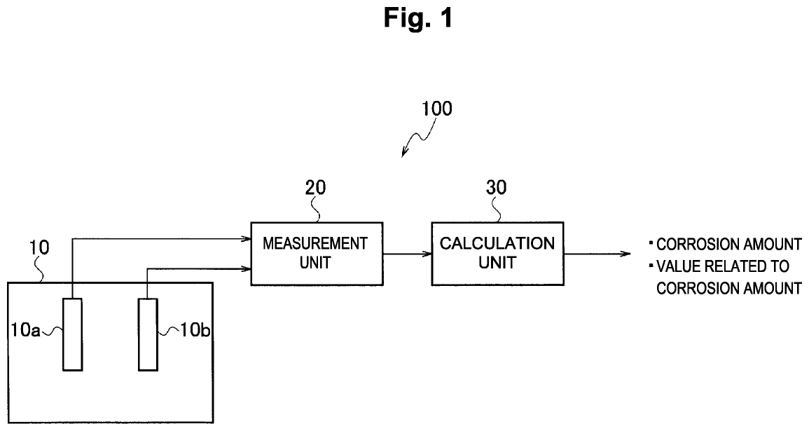

FIG. 1 is a block diagram showing an exemplary functional configuration of a corrosivity evaluation device according to an embodiment of the present invention. The corrosivity evaluation device 100 shown in FIG. 1 evaluates the corrosivity that represents the extent to which metal is corroded by an environment. The corrosivity is corrosivity of the environment.

The corrosivity of the environment is, for example, a property of the soil in which infrastructure equipment is placed, the property representing the extent to which the soil corrodes the equipment. For example, if the equipment is corroded quickly, it is said that the soil is high in corrosivity, and if the equipment is corroded slowly, it is said that the soil is low in corrosivity. The corrosivity evaluation device 100 quantitatively evaluates the corrosivity level of the environment in which the infrastructure equipment is placed.

In FIG. 1, illustration of the environment is omitted. The environment may be any of soil, water, and atmospheric environments. In the following description, a soil environment is taken as an example.

The corrosivity evaluation device 100 comprises an electrode unit 10, measurement unit 20, and a calculation unit 30. The electrode unit 10 includes two or more metal pieces placed in the environment by being spaced away from each other.

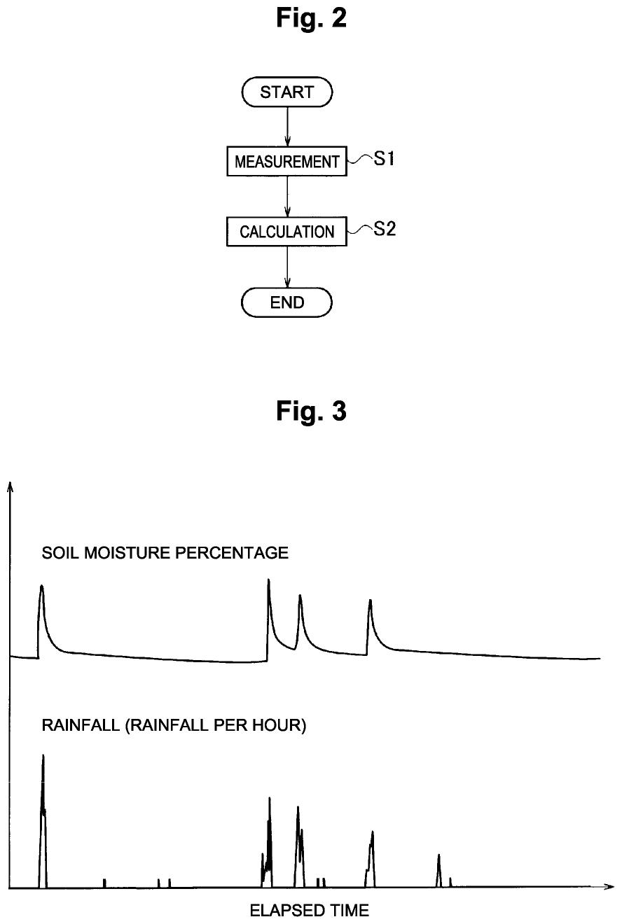

FIG. 2 is a flowchart showing processing procedures of the corrosivity evaluation device 100. Operation of the corrosivity evaluation device 100 will be described with reference to FIGS. 1 and 2.

The electrode unit 10 shown in FIG. 1 is an example in which two metal strips (metal pieces 10a and 10b) to be evaluated are placed in the environment. The metal pieces 10a and 10b are made of the same type of metal. That is, the electrode unit 10 contains at least one type of metal by being placed in the environment.

In the example shown in FIG. 1, the electrode unit 10 is buried in the soil to be evaluated. Note that there is no particular limit to the shape of the metal pieces 10a and 10b including size and thickness.

From one cycle of change in water content of the environment, the measurement unit 20 measures corrosion rates of the metal pieces 10a and 10b or values related to the corrosion rates of the metal pieces 10a and 10b during the change (step S1). The one cycle of change in water content means, for example, changes in soil moisture percentage between 100% and 0%. Note that the upper limit is not necessarily 100% and the lower limit is not necessarily 0%.

The one cycle of change in water content of the environment can be grasped by appropriately setting intervals and a period of corrosion rate measurements. For example, in the case of well-drained soil, a corrosion rate corresponding to one cycle of change in water content can be measured at measurement intervals of about a few hours for a measurement period of about one day.

In this example, the environment is the soil. The soil is a mixed three-phase environment made up of soil particles and a gas phase and liquid phase (water) existing among the soil particles, where the soil particles are made of oxides of Si, Al, Ti, Fe, Ca, and the like. The sum total of the proportions of the gas phase and liquid phase in the soil can be regarded to be constant, the two phases being in a reciprocal relationship in which when one of the phases increases, the other decreases. Also, a corrosion reaction in the soil basically requires water and oxygen, and corrosion progresses at a corrosion rate dependent on conditions of water and oxygen.

Thus, soil moisture percentage, which means a proportion of water in soil, is an important environmental factor contributing to the corrosion rate, and it can be said that the corrosion rate changes with the soil moisture percentage.

The soil moisture percentage is not always kept constant unless at a position very deep underground. The soil moisture percentage changes, for example, with natural phenomena such as rainfall.

FIG. 3 is a diagram schematically showing a relationship between rainfall and soil moisture percentage. The abscissa in FIG. 3 represents elapsed time. As shown in FIG. 3, soil moisture percentage increases and decreases in close connection with rainfall, repeating cycles of increasing suddenly during rainfall and decreasing gradually when the rain stops. Thus, it can be considered that changes in corrosion rate over time also repeat cycles beginning with rainfall.

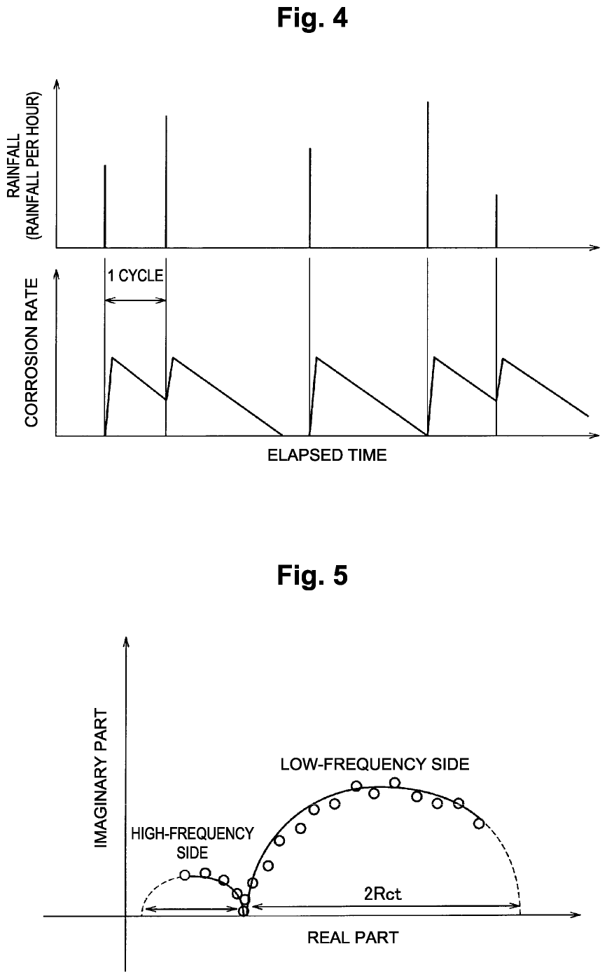

FIG. 4 is a diagram schematically showing a relationship between rainfall and a corrosion rate of metal in soil. Here, one cycle means a period from rainfall to next rainfall. The time length of one cycle varies with the rainfall interval.

Note that besides the soil moisture percentage, there are many factors contributing to the corrosion rate. Examples of such factors include a pH value and various ion contents. Because basically these ion species have leached from soil into water, once the soil and moisture percentage are determined, the pH value and the various ion contents are determined uniquely. Thus, it can be considered that time variations of these factors also change cyclically beginning with rainfall.

The measurement unit 20 measures the corrosion rates of the metal pieces 10a and 10b or values related to the corrosion rates of the metal pieces 10a and 10b during the one cycle of change in soil moisture percentage. A concrete measuring method will be described later. Note that while the measurement unit 20 measures the corrosion rates and the like of the metal pieces 10a and 10b of the electrode unit 10, the corrosion rates are determined depending on interaction with the environment in which the metal pieces 10a and 10b are placed. Thus, the corrosion rates and the like measured by the measurement unit 20 represent the corrosivity level of the environment.

The calculation unit 30 calculates corrosion amounts of the metal pieces 10a and 10b or values related to the corrosion amounts of the metal pieces 10a and 10b from the values measured by the measurement unit 20 (step S2). From the corrosion rates or the values related to the corrosion rates measured during the one cycle of change in soil moisture percentage, the calculation unit 30 calculates the corrosion amounts or the values related to the corrosion amounts. The calculated values may be output directly to the outside or may be compared with some reference values to determine the degree of corrosivity. As described above, the degree of corrosivity represents the corrosivity of the environment in which the metal pieces 10a and 10b are placed.

As has been described above, the corrosivity evaluation device 100 according to the present embodiment is an device that evaluates corrosivity that represents an extent to which the metal pieces 10a and 10b are corroded by an environment, the device comprising: the electrode unit 10 including two or more metal pieces 10a and 10b placed in the environment by being spaced away from each other; the measurement unit 20 adapted to measure the corrosion rates of the metal pieces 10a and 10b or values related to the corrosion rates of the metal pieces 10a and 10b during one cycle of change in the water content of the environment, the measurements being taken from the one cycle of change; and the calculation unit 30 adapted to calculate corrosion amounts of the metal pieces 10a and 10b or values related to the corrosion amounts of the metal pieces 10a and 10b from the values measured by the measurement unit 20. This makes it possible to quantitatively evaluate the corrosivity of the environment. The corrosivity of the environment can be found from one cycle of change in water content. Thus, the corrosivity of the environment can be evaluated quantitatively in a short time.

Next, functional components of the corrosivity evaluation device 100 will be described in detail.

### Electrode Unit

The electrode unit 10 needs to have as many electrodes as necessary for electrochemical measurements conducted by the measurement unit 20. For example, for AC impedance measurement using a two-electrode method, the electrode unit 10 is equipped with the metal pieces 10a and 10b as shown in FIG. 1.

The metal pieces 10a and 10b are buried directly in the soil to be evaluated. Note that corrosivity may be evaluated by taking a sample of the soil to be evaluated and inserting the metal pieces 10a and 10b into the soil sample. An evaluation method using a soil sample will be described later.

For AC impedance measurement using a three-electrode method, a working electrode, counter electrode, and reference electrode are provided. In this case, platinum, a carbon sheet, or the like is used for the counter electrode, and an Ag/AgCl electrode, copper sulfate electrode, or the like is used as the reference electrode. Note that AC impedance measurement using a three-electrode method is commonly known.

### Measurement Unit

The measurement unit 20 has an AC impedance measurement function. The AC impedance measurement involves using metal pieces placed in an environment as electrodes, applying a micro AC voltage or micro AC current between the electrodes, and measuring electrical responses. Note that the metal pieces are not limited to the two metal pieces 10a and 10b described above.

It is advisable that the voltage or current applied to the metal is so weak as not to cause changes to metal surfaces. For example, the voltage applied is about +/−5 mV. The frequency is varied, for example, in a range of 0.1 Hz to a few kHz.

By measuring AC impedance, a Nyquist diagram can be obtained. A Nyquist diagram is shown schematically in FIG. 5. The abscissa of the Nyquist diagram represents real part and the ordinate represents imaginary part. Using the Nyquist diagram and based on a predetermined equivalent circuit, charge transfer resistance is derived through curve fitting.

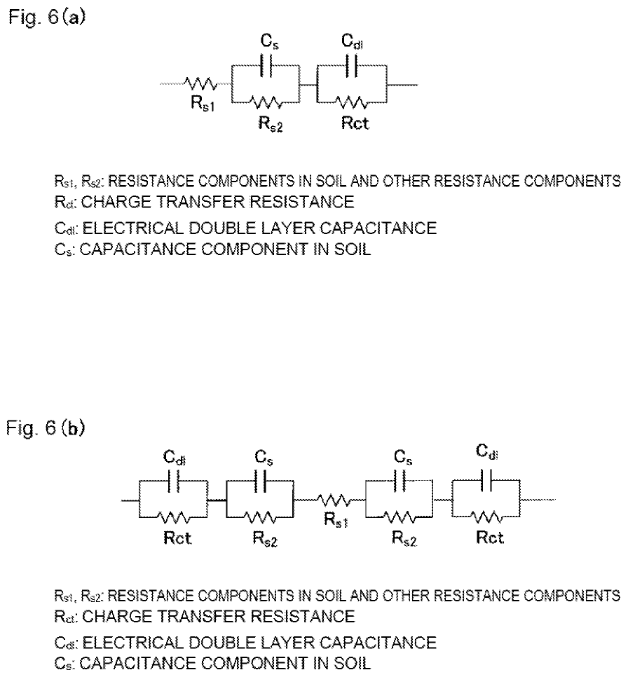

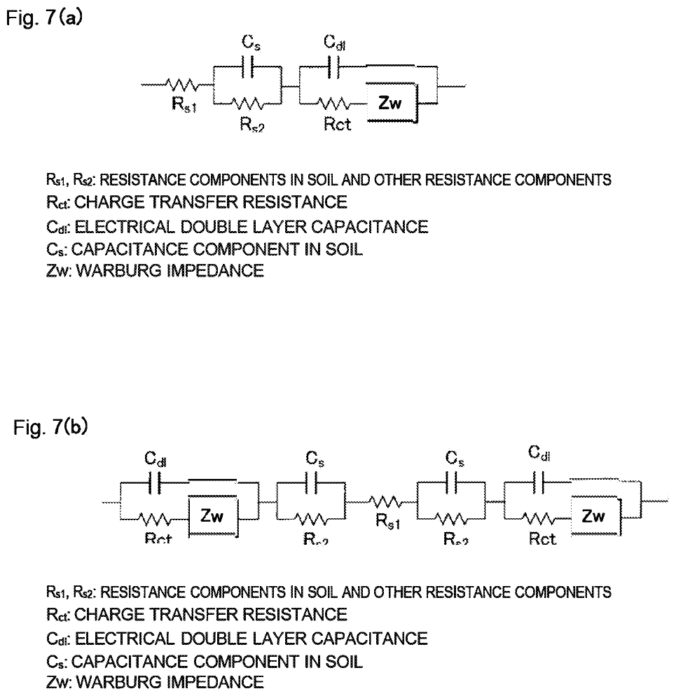

FIGS. 6(a) to 7(b) are diagrams showing examples of equivalent circuits assumed in calculating charge transfer resistance. FIGS. 6(a) and 7(a) are equivalent circuits measuring AC impedance using three electrodes. FIGS. 6(b) and 7(b) are equivalent circuits measuring AC impedance using two electrodes.

Charge transfer resistance Rct in the figures is resistance of a corrosion reaction of the metal buried in the soil. An electrical double layer C_{dl} provides capacitance existing in an interface between the metal and soil. Resistance components Rₛ₁ and Rₛ₂ are resistance of the soil and other resistance. Capacitance Cₛ is a capacitance component of the soil. Warburg impedance Z_{w} (FIG. 7) is impedance caused by a diffusion process. Note that in curve fitting, the electrical double layer C_{dl} and capacitance Cₛ may be substituted with a CPE (Constant Phase Element).

The equivalent circuits shown in FIGS. 6(a) to 7(b) theoretically make two circular arcs drawn on a Nyquist diagram as shown in FIG. 5. The circular arc on the high-frequency side originates in the soil. The circular arc on the low-frequency side originates in the corrosion reaction.

The charge transfer resistance Rct is given by the width over which the circular arc on the low-frequency side of the Nyquist diagram intersects the abscissa (real part). Note that when AC impedance is measured using two electrodes, the charge transfer resistance Rct is given by half the width.

The corrosion rate is proportional to the inverse of the charge transfer resistance Rct. The corrosion rate is synonymous with an amount of ionization on a unit area of a metal surface per unit time, i.e., with current density. Corrosion current density is found using the inverse of the charge transfer resistance Rct derived from the principle of polarization resistance known as the Stern-Geary equation and a proportionality constant K (Reference: “Corrosion Monitoring of Metals in Soils by Electrochemical and Related Methods: Part 2,” Zairyo-to-Kankyo, 1967, Vol. 46, pp. 610-619).

The proportionality constant K may be found experimentally. The proportionality constant K is found in advance from results of an anode polarization test and cathode polarization test of metal in soil of interest.

The use of the proportionality constant K allows the corrosion current density (corrosion rate) to be calculated from the inverse of the charge transfer resistance Rct. Also, a weight loss rate, volume loss rate, or another value related to the corrosion rate may be calculated from corrosion current density.

In this way, from a result of one impedance measurement taken in the measurement step (step S1), one corrosion rate or a value (1/Rct) proportional to one corrosion rate can be obtained.

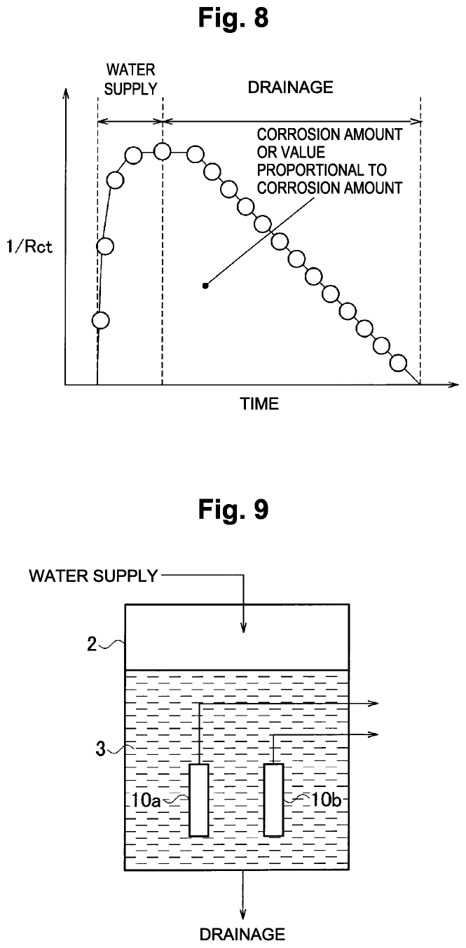

FIG. 8 is a diagram schematically showing a relationship between time corresponding to one cycle of water supply and drainage, during which changes in water content occur, and a value (1/Rct) proportional to a corrosion rate. In FIG. 8, the abscissa represents the time corresponding to one cycle of water supply and drainage, during which changes in water content occur, and the ordinate represents the value (1/Rct) proportional to a corrosion rate.

The measurement unit 20 measures the charge transfer resistance Rct every predetermined time. The time required for one cycle varies depending on whether drainage characteristic of the soil of interest is good or poor. For example, the time required for one cycle may be a few hours, or a period on the order of days if the soil is poorly drained and always wet. Also, the predetermined time may be set as desired, but is desirably adjusted according to the drainage of the soil because preferably plural measurements are taken in one cycle.

If the predetermined time is assumed, for example, to be one hour, the measurement unit 20 finishes the measurement of the charge transfer resistance Rct shown in FIG. 8 in 18 hours. From the measured charge transfer resistance Rct, the measurement unit 20 may calculate the corrosion rate (corrosion current density) or calculate the weight loss rate or volume loss rate.

### Calculation Unit

From the corrosion current density (corrosion rate) or weight loss rate or another value measured by the measurement unit 20, the calculation unit 30 finds the corrosion amount of metal or a value related to the corrosion amount. The corrosion amount of metal or a value related to the corrosion amount thus found is output to the outside.

The calculation unit 30 fits the time variation of the corrosion rate or value proportional to the corrosion rate to a function f(t) and finds an integral of the function f(t) as a corrosion amount. From the magnitude of the corrosion amount thus found, the corrosivity of soil (environment) is able to be evaluated.

As has been described above, the corrosivity evaluation method according to the present embodiment includes a measurement step (S1) of measuring, for example, corrosion rates of the metal pieces 10a and 10b or, for example, values related to the corrosion rates of the metal pieces 10a and 10b during one cycle of change in water content of an environment in which two or more metal pieces are placed, the measurements being taken from the one cycle of change; and a calculation step (S2) of calculating corrosion amounts of the metal pieces or values related to the corrosion amounts of the metal pieces from the values measured in the measurement step. This makes it possible to quantitatively evaluate the corrosivity of the environment in a short time.

An example of corrosion amounts found by the corrosivity evaluation device 100 is shown in Table 1.

TABLE 1

| Soil | Corrosion amount |
|---|---|
| (1) | 0.004 |
| (2) | 0.012 |
| (3) | 0.006 |
| (4) | 0.022 |

Soil (1) is red soil, soil (2) is gray lowland soil, soil (3) is black soil, and soil (4) is peat soil. The corrosion amount is calculated by multiplying the value (1/Rct) proportional to the corrosion rate by time.

If the corrosion amounts calculated are as shown in Table 1, the corrosivity decreases in the order: (4)>(2)>(3)>(1). The corrosivity may be evaluated using quantitative values as shown in Table 1 or evaluated by providing a reference and comparing with the reference.

For example, the corrosivity evaluation device 100 may include an evaluation unit (not shown) adapted to accept as input the corrosion amounts and the like calculated by the calculation unit 30 and may determine that the soil has corrosivity if a reference value managed by the evaluation unit is exceeded and determine that the soil does not have corrosivity if the reference value is not reached. In the example shown in Table 1, the reference value can be, for example, 0.010.

Note that instead of comparing the found value itself, the corrosion amount or a value proportional to the corrosion amount may be converted into another evaluation reference value. For example, by designating the corrosion amount or a value proportional to the corrosion amount as x based on a certain evaluation reference, an evaluation value g(x) may be found.

### Evaluation Method Using a Soil Sample

After a soil sample to be evaluated is obtained and contained in a container unit, the metal pieces 10a and 10b may be buried in the soil sample to evaluate corrosivity.

FIG. 9 is a diagram schematically showing how a soil sample 3 is contained in a container unit 2 and the metal pieces 10a and 10b are buried in the soil sample 3. Water may be supplied to the container unit 2 from a non-illustrated water supply mechanism. A soil sample kept at a predetermined soil moisture percentage in advance may be used alternatively.

Water in the soil sample 3 is discharged to the outside from lower part of the container unit 2. A simple drain mechanism can be implemented by installing a porous filter in lower part of the container unit 2.

Note that it is sufficient if the water supply mechanism and drain mechanism can change the soil moisture percentage of the soil sample 3, and implementation form and method of the mechanisms do not matter. For example, water may be supplied to the soil sample 3 manually.

Also, the container unit 2 may include an environmental function part configured to simulate the environment to be evaluated. Conceivable examples of the environmental function part include a temperature control function part (not shown) and oxygen concentration control function part.

The temperature control function part is, for example, a constant temperature bath, and when the container unit 2 is put in the constant temperature bath, the temperature of the environment to be evaluated can be simulated.

The oxygen concentration control function part can be implemented by providing a space in the container unit 2 to expose a surface of the soil sample 3 to gas. By providing an inlet port for use to introduce gas into the space and an outlet port for use to discharge the gas, for example, a gas mixture of N₂ and O₂ are introduced. Also, CO₂ may be added.

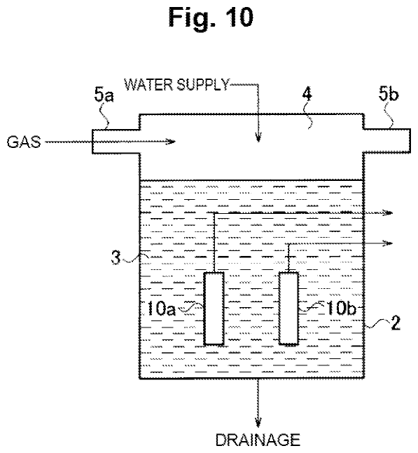

FIG. 10 is a diagram schematically showing an example of the container unit 2 provided with a space 4 configured to expose the surface of the soil sample 3 to a predetermined gas. The gas is introduced through an inlet port 5a and discharged through an outlet port 5b. If the gas used here is the gas mixture described above and a mixing ratio of the gases is varied, oxygen concentration in the soil sample 3 can be controlled. That is, the space 4, inlet port 5a, and outlet port 5b shown in FIG. 10 make up the oxygen concentration control function part. This makes it possible to create a simulated environment close to an actual soil environment and thereby improve reliability of corrosivity evaluation.

In this way, the corrosivity evaluation device 100 according to the present embodiment may include the container unit 2. Note that although an example of containing a soil sample in the container unit 2 has been described, the container unit 2 is not limited to this example. The container unit 2 may contain only gas or contain two phases of liquid and gas. When only gas is contained, the soil moisture percentage described above equals humidity in the container unit 2.

Thus, the water content of an environment is not limited to soil moisture percentage. When, for example, two phases of liquid and gas are contained in the container unit 2, the water content of the environment means the proportion (amount) in which the metal pieces 10a and 10b are immersed in the liquid or the frequency at which surfaces of the metal pieces 10a and 10b are exposed to the liquid, and the like. That is, one cycle of change in water content of the environment means one cycle of change in quantities related to water, such as water content, water film thickness, and humidity on surfaces of the metal placed in the environment.

The container unit 2 encloses an environment simulating the environment whose corrosivity is to be evaluated. That is, the corrosivity evaluation device 100 includes the container unit 2 configured to contain the electrode unit 10. From one cycle of change in moisture percentage in the container unit 2, the measurement unit 20 measures, for example, corrosion rates of the metal pieces 10a and 10b or, for example, values related to the corrosion rates of the metal pieces 10a and 10b during the change. This makes it possible to evaluate the corrosivity of the environment by staying in a laboratory.

As has been described above, the corrosivity evaluation device 100 according to the present embodiment can quantitatively evaluate the corrosivity of the environment. Note that although in the above embodiment, the environment has been described by taking soil as an example, the present invention is not limited to this example.

The environment may be an atmospheric environment or aqueous environment. By placing the electrode unit 10 in such an environment, the corrosivity of the environment can be evaluated quantitatively with accuracy in line with the actual situation.

The present invention is not limited to the embodiments described above, and changes can be made within the scope of the invention. For example, although description has been given of a case in which the electrode unit 10 is made up of two metal pieces 10a and 10b placed by being spaced away from each other, the electrode unit may include three electrodes of a counter electrode, working electrode, and reference electrode.

Thus, needless to say, the present invention includes various embodiments and the like not described herein. Therefore, the technical scope of the present invention is defined only by matters specifying the invention that are set forth in the scope of claims appropriate from the above description.

### Reference Signs List

100 Corrosivity evaluation device
2 Container unit
3 Soil sample
4 Space (environmental function part)
10 Electrode unit
10a, 10b Metal piece
20 Measurement unit
30 Calculation unit

1. A corrosivity evaluation device that evaluates corrosivity that represents an extent to which metal is corroded by an environment, the device comprising:
an electrode unit containing at least one type of the metal by being placed in the environment;
a measurement unit adapted to measure a corrosion rate of the metal or a value related to the corrosion rate of the metal during one cycle of change in water content of the environment, the measurements being taken from the one cycle of change; and
a calculation unit adapted to calculate a corrosion amount of the metal or a value related to the corrosion amount of the metal from the value measured by the measurement unit.

2. The corrosivity evaluation device according to claim 1, further comprising a container unit configured to contain the electrode unit, wherein
the measurement unit measures the corrosion rate of the metal or a value related to the corrosion rate of the metal during one cycle of change in moisture percentage in the container unit, the measurements being taken from the one cycle of change.

3. The corrosivity evaluation device according to claim 2, wherein the container unit includes an environmental function part configured to simulate the environment to be evaluated.

4. A corrosivity evaluation method performed by a corrosivity evaluation device that evaluates corrosivity that represents an extent to which metal is corroded by an environment, the method comprising:
a measurement step of measuring a corrosion rate of the metal or a value related to the corrosion rate of the metal during one cycle of change in water content of an environment in which at least one type of the metal is placed, the measurements being taken from the one cycle of change; and
a calculation step of calculating a corrosion amount of the metal or a value related to the corrosion amount of the metal from the value measured in the measurement step.
